(19) United States
(12) Patent Application Publication
(10) Pub. No.: US 2025/0073280 A1
(43) Pub. Date: Mar. 6, 2025

(54) CORYNEBACTERIUM TO TREAT OR LIMIT CUTANEOUS WOUND INFECTION

(71) Applicants: MASSACHUSETTS INSTITUTE OF TECHNOLOGY, Cambridge, MA (US); THE GENERAL HOSPITAL CORPORATION, Boston, MA (US)

(72) Inventors: Tami Danielle LIEBERMAN, Cambridge, MA (US); Victor NEEL, Boston, MA (US); Alexandra Jeena PORET, Cambridge, MA (US)

(21) Appl. No.: 18/726,542

(22) PCT Filed: Jan. 6, 2023
(86) PCT No.: PCT/US2023/060228

§ 371 Date: Jul. 3, 2024

Related U.S. Application Data
(60) Provisional application No. 63/297,365, filed on Jan. 7, 2022.

(51) Int. Cl. A61K 35/741; A61K 31/201; A61K 31/7004; A61K 31/7048; A61K 38/48; A61K 45/06; A61K 47/10; A61K 47/36; A61P 31/04

(52) U.S. Cl. CPC A61K 35/741; A61K 31/201; A61K 31/7004; A61K 31/7048; A61K 38/4886; A61K 45/06; A61K 47/10; A61K 47/36; A61P 31/04; C12Y 304/24075

(57) ABSTRACT

Methods and compositions for treating a cutaneous wound to treat or limit development of a pathogenic bacterial infection are provided, involving administering to a subject having a cutaneous wound an amount effective of Corynebacteria spp., or a disclosed, composition, to treat or limit development of pathogenic bacterial infection of the wound.

### REFERENCE TO RELATED APPLICATIONS

This application claims priority to U.S. Provisional Patent Application Ser. No. 63/297,365, filed Jan. 7, 2022, incorporated by reference herein in its entirety.

### BACKGROUND OF THE INVENTION

The structural integrity of skin presents a formidable barrier to invasion by pathogenic bacteria commonly encountered in the environment. It is generally accepted that following the disruption of this barrier—due to surgery, abrasion, or other insults—the innate and adaptive arms of the immune system protect against infection until the barrier is re-established. However, it is increasingly recognized that the skin microbiome plays a critical role in skin homeostasis, and it has long been hypothesized that disturbances to the skin flora are associated with susceptibility to pathogens. However, the specific role of commensal microbes in promoting infection-free recovery after injury is poorly understood.

### SUMMARY OF THE INVENTION

In one aspect, the disclosure provides methods for treating a cutaneous wound to treat or limit development of a pathogenic bacterial infection, comprising administering to a subject having a cutaneous wound an amount effective of Corynebacteria spp. to treat or limit development of pathogenic bacterial infection of the wound. In one embodiment, the method treats or limits development of a pathogenic Staphylococcal infection of the wound, including but not limited to a Staphylococcal aureus infection of the wound. In another embodiment, the cutaneous wound is selected from the group consisting of a penetrating wound, a puncture wound, a surgical wound or incision, a skin ulceration, a burn including but not limited to a thermal, chemical or electric burn; an insect bite or sting, a gunshot wound, or a wound caused by other high velocity projectile. In one embodiment, the administering occurs while the wound is open, and/or after wound healing has commenced. In various embodiments, the Corynebacteria spp. comprise one or more of C. tuberculostearicum, C. accolens, C. striatum, C. amycolatum, and/or C. pseudodipthericum. In another embodiment, the Corynebacteria spp. is heat inactivated, UV radiation inactivated, antibiotic treatment inactivated, and/or chloroform inactivated. In a further embodiment, the Corynebacteria spp. are topically administered.

In another aspect, the disclosure provides composition comprising Corynebacteria spp. In one embodiment, the composition is formulated for topical administration. In some embodiments, the formulation is selected from the group consisting of hydrogels, foams, creams, ointments, pastes, and lotions. In other embodiments, the composition is present on a wound dressing, including but not limited to semipermeable films, foams, hydrocolloids, and calcium alginate swabs. In one embodiment, the Corynebacteria spp. are dehydrated and/or freeze dried. In various embodiments, the Corynebacteria spp. comprise one or more of C. tuberculostearicum, C. accolens, C. striatum, C. amycolatum, and/or C. pseudodipthericum. In other embodiments, the Corynebacteria spp. is heat inactivated, UV radiation inactivated, antibiotic treatment inactivated, and/or chloroform inactivated. In a further embodiment, the Corynebacteria spp. are present in the composition at between about 10⁴ and about 10¹² colony forming units (cfu). In another embodiment, the disclosure provides kits comprising the composition of any embodiment disclosed herein.

### BRIEF DESCRIPTION OF THE FIGURES

FIG. 1. After surgery, the healthy wound microbiota is disrupted, characterized by a loss of Cutibacterium. (A) Comparing the average Bray-Curtis or Unifrac dissimilarity within control samples (labeled C-C), within surgical samples (labeled S-S). or between control and surgical samples (labeled C-S) displays that microbiome samples from control skin are more similar to one another than wound-normal or wound-wound pairs, p-value: *=<10⁻⁶. The fraction of Cutibacterium seen in every contralateral control and surgical sample one week post-surgery is depicted, displaying significant differences in abundance (Wilcoxon rank sum, p<10⁻⁶). (B) Bar graphs displaying the genus-level composition of all contralateral control and surgery samples sorted by descending Cutibacterium abundance visually depicts Cutibacterium depletion.

FIG. 2. S. aureus asymptomatically colonizes healthy surgical wounds from the surrounding skin microbiota. (A) The number of patients that contained S. aureus on their day ˜7 and day 0 surgical site as well as on any sampled day ˜7 and day 0 control sites are shown (p=0.01, Fisher's Exact test). (B) The fraction of S. aureus, S. epidermidis, and S. capitis for patient-and-site-matched control and surgical samples one week post-surgery is shown. A Wilcoxon-sign rank indicates a significant increase in S. aureus (p=0.002), a decrease S. epidermidis (p=0.03), and a decrease in S. capitis (p<10⁻⁶).

FIG. 3. Specific Corynebacterium species are enriched in healthy healing skin wounds. (a) A phylogenetic tree created from the 16S rRNA gene of all Corynebacterium species observed in matched surgical-control samples is shown. Darker bars indicate the average abundance observed in control samples and lighter bars indicate the average abundance observed in surgical samples. (b) The mean Corynebacterium abundance of all contralateral control and week-post-surgery samples is shown in the left graph (Wilcoxon sign rank. p=0.004). The right depicts the average Corynebacterium fraction after removing all Cutibacterium reads and renormalizing total abundance to 1 (Wilcoxon rank sum=0.02).

FIG. 4. Graph showing C. tuberculostearicum inhibits S. aureus growth in solid media. Lines represent means, dots represent individual experiments.

FIG. 5. Graph showing C. tuberculostearicum inhibits RNAIII expression normalized to phosphate acetyltransferase (PTA) expression. Lines represent means, dots represent individual experiments.

### DETAILED DESCRIPTION

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural or singular number, respectively. Additionally, the words “herein,” “above” and “below” and words of similar import, when used in this application, shall refer to this application as a whole and not to any particular portions of this application.

All embodiments of any aspect of the invention can be used in combination, unless the context clearly dictates otherwise.

As used herein. “about” means+/−5% of the recited value.

In a first aspect, the disclosure provides methods for treating a cutaneous wound to treat or limit development of a pathogenic bacterial infection, comprising administering to a subject having a cutaneous wound an amount effective of Corynebacteria spp. to treat or limit development of pathogenic bacterial infection of the wound. The methods may be used to treat or limit development of any pathogenic bacterial infection of the wound. In one embodiment, the pathogenic bacteria is not a Corynebacteria spp. In another embodiment. the method treats or limits development of a pathogenic Staphylococcal infection of the wound, including but not limited to a Staphylococcal aureus infection of the wound.

As described herein, the inventors have determined that in naturally-healing (non-sutured) surgical wounds in humans, multiple Corynebacterial species rapidly colonize open wounds. These wounds are clinically normal (not infected) and are also sometimes. surprisingly. colonized with pathogenic bacteria, including but not limited to S. aureus, which is usually considered a pathogen. However, wounds that are co-colonized with Corynebacteria show no clinical evidence of infection and heal normally. Thus, the methods of the disclosure can treat a cutaneous wound to treat or limit development of a bacterial infection, including but not limited to a Staphylococcal infection.

Symptoms associated with bacterial infections (including but not limited to Staphylococcal aureus infections), include, but are not limited to, fever, nausea, boils, impetigo or other rashes, cellulitis, Staphylococcal scalded skin syndrome, toxic shock syndrome, and/or bacteremia.

As used herein, “treating” a bacterial infection of the wound means providing any treatment benefit to the subject. In various embodiments, such benefits include: (a) reducing or eliminating the infection, (b) slowing an increase of the infection; (c) reducing or eliminating symptoms of the infection: (d) limiting worsening of symptoms of the infection; and/or (e) reducing time to recovery from the infection.

As used herein. “limiting development of” a bacterial infection of the wound means providing any benefit to a subject that does not yet have a bacterial infection, including but not limited to a Staphylococcal aureus infection. In various embodiments, such benefits include (a) preventing pathogenic bacterial infection of the wound, (b) slowing pathogenic bacterial infection of the wound; and/or (c) slowing development of symptoms associated with pathogenic bacterial infection of the wound.

In embodiments where the pathogenic bacterial infection comprises a Staphylococcal aureus infection, the infection may comprise Staphylococcal aureus only, or may comprise one or more other pathogenic bacteria and/or Staphylococcal spp. in addition to Staphylococcal aureus.

In one embodiment, the Corynebacteria spp. may be autologously engrafted from a high-density site Corynebacteria spp. on the subject to the wound site. Anatomic areas of the body with naturally high levels of Corynebacteria within the skin microbiome, including but not limited to the face, have a lower rate of infection when compared to anatomic areas with naturally low levels of Corynebacteria, including but not limited to the lower legs. In other embodiments, the Corynebacteria spp. are provided as a separate composition for treatment, as described in more detail below.

As used herein, a cutaneous wound is any wound that penetrates the first layer of skin. In various non-limiting embodiments, the cutaneous wound may comprise a penetrating wound, a puncture wound, a surgical wound or incision, a skin ulceration, a burn including but not limited to a thermal, chemical or electric burn; an insect bite or sting, a gunshot wound, a wound caused by other high velocity projectile, or a wound caused by trauma. In one specific embodiment, the cutaneous wound comprises a surgical wound.

The cutaneous wound may be on any area of the subject's skin, including but not limited to arms, hands, feet, legs, face, back, and torso. In one embodiment, the cutaneous wound is in an anatomic area with naturally low levels of Corynebacteria, including but not limited to the lower leg (i.e.: below the knee).

The Corynebacteria spp. may be administered at the time of or after cutaneous wounding, as deemed appropriate. In one embodiment, the administering occurs while the wound is open, in that it has not been sutured closed. In another embodiment, the administering occurs after wound healing has commenced, but prior to closing of the wound by natural healing processes. In a further embodiment, the administering occurs at the time of suturing. In other embodiments, the administering is done to a wound that will be left open and not sutured, including but not limited to a subject undergoing Mohs surgery.

Any Corynebacteria spp. may be used as deemed appropriate by attending medical personnel. In one embodiment the Corynebacteria spp. comprises one or more Corynebacteria species selected from the group consisting of C. tuberculostearicum, C. accolens. C. striatum. C. amycolatum, and/or C. pseudodipthericum. In another embodiment, the Corynebacteria spp. comprises one or more Corynebacteria species selected from the group consisting of C. tuberculostearicum, C. accolens. C. amycolatum, and/or C. pseudodipthericum. In another embodiment, the Corynebacteria spp. comprises one or more Corynebacteria species selected from the group consisting of C. tuberculostearicum. C. accolens, and/or C. pseudodipthericum. In another embodiment, the Corynebacteria spp. comprises one or more Corynebacteria species selected from the group consisting of C. tuberculostearicum and/or C. pseudodipthericum. In another embodiment, the Corynebacteria spp. comprises one or more Corynebacteria species or strains capable of inhibiting argA signaling in S. aureus.

The Corynebacteria spp. may be administered to the wound in any amount deemed suitable by attending medical personnel to treat or limit development of a Staphylococcal aureus infection. In one non-limiting embodiment, the methods comprise administering between about 10⁴ and about 10¹² colony forming units (cfu) of Corynebacteria spp. The administering may comprise a single administration or multiple administrations per day or over any other period of time as deemed appropriate by attending medical personnel.

The Corynebacteria spp. may be administered to the cutaneous wound via any administrative route deemed suitable by attending medical personnel. In one embodiment, the Corynebacteria spp. are topically administered. In one embodiment, the topical administration comprises administration in a formulation selected from the group consisting of hydrogels, foams, creams, ointments, pastes, and lotions. The formulations may be applied in any suitable manner, which may include any wound dressings to seal in the formulation deemed appropriate by the human patient or caregiver. Exemplary such dressings, include, but are not limited to, semipermeable films, foams, hydrocolloids, and calcium alginate swabs. In another embodiment, the topical administration comprises administration in a formulation comprising a tissue glue, including but not limited to cyanoacrylate.

The Corynebacteria spp. may be modified in any way as deemed appropriate by attending medical personnel. In one embodiment, Corynebacteria spp. may be inactivated to be live but replication deficient, via any suitable method. including but not limited to heat inactivation, UV irradiation, antibiotics, and/or chloroform treatment. In another embodiment, the Corynebacteria spp. is genetically modified to inactivate one or more virulence factors and/or resistance cassettes. Non-limiting examples of such resistance cassettes include erythromycin, clindamycin, penicillins (class), ciprofloxacin, and trimethoprim-sulfamethoxazole.

In a further embodiment, the Corynebacteria spp. may be dehydrated or freeze dried and reconstituted prior to administering to the subject.

The Corynebacteria spp. may be the sole active agent administered, or the methods may further comprise one or more other active agents as deemed appropriate by attending medical personnel. In one embodiment, the methods further comprise administering antimicrobials to which Corynebacterium is resistant but S. aureus is sensitive, including but not limited to lysostaphin and mupirocin. In another embodiment, the methods further comprise administering compounds that benefit growth of Corynebacterium or specific species of Corynebacterium, including but not limited to oleic acid and/or fructose.

The Corynebacteria spp. may be administered in a pharmaceutical composition that includes any other components as deemed appropriate. In one embodiment, the composition comprises one or both of 10-50% glycerol and/or chitosan.

The subject may be any subject that may benefit from the methods, including but not limited to a human subject.

In another aspect, the disclosure provides compositions, comprising Corynebacteria spp. All embodiments disclosed in the first aspect of the disclosure are applicable to the compositions of the second aspect. In one embodiment, the composition is formulated for topical administration. In certain embodiments, the formulation is selected from the group consisting of hydrogels, foams, creams, ointments, pastes, and lotions. The formulations may, for example, be placed on wound dressings to seal in the formulation deemed appropriate by the human patient or caregiver. Exemplary such dressings, include, but are not limited to, semipermeable films, foams. hydrocolloids, and calcium alginate swabs.

In another embodiment, the Corynebacteria spp. may be inactivated to be live but replication deficient, via any suitable method, including but not limited to heat inactivation, UV radiation, antibiotics, and/or chloroform treatment. In another embodiment, the Corynebacteria spp. is genetically modified to inactivate one or more virulence factors and/or resistance cassettes. Non-limiting examples of such resistance cassettes include erythromycin, clindamycin, penicillins (class), ciprofloxacin, and trimethoprim-sulfamethoxazole.

In a further embodiment, the Corynebacteria spp. may be dehydrated/freeze dried, but reconstitutable prior to use.

The Corynebacteria spp. may be the sole active agent in the composition, or the composition may further comprise one or more other active agents. In one embodiment, the composition further comprises antimicrobials to which Corynebacterium is resistant but S. aureus is sensitive, including but not limited to lysostaphin and mupirocin. In another embodiment, the composition further comprises one or more compounds that benefit growth of Corynebacterium, including but not limited to oleic acid and/or fructose.

The composition may be present in a pharmaceutical composition or probiotic that includes any other components as deemed appropriate. In one embodiment, the composition comprises one or both of 10-50% glycerol and/or chitosan.

The compositions may comprise any Corynebacteria spp. In one embodiment the Corynebacteria spp. comprises one or more Corynebacteria species selected from the group consisting of C. tuberculostearicum, C. accolens, C. striatum, C. amycolatum, and/or C. pseudodipthericum. In another embodiment, the Corynebacteria spp. comprises one or more Corynebacteria species selected from the group consisting of C. tuberculostearicum, C. accolens, C. amycolatum, and/or C. pseudodipthericum. In another embodiment, the Corynebacteria spp. comprises one or more Corynebacteria species selected from the group consisting of C. tuberculostearicum. C. accolens, and/or C. pseudodipthericum. In another embodiment, the Corynebacteria spp. comprises one or more Corynebacteria species selected from the group consisting of C. tuberculostearicum and/or C. pseudodipthericum. In another embodiment, the Corynebacteria spp. comprises one or more Corynebacteria species or strains capable of inhibiting argA signaling in S. aureus.

The Corynebacteria spp. may be present in the composition in any suitable amount. In one non-limiting embodiment, the composition comprises between about 10⁴ and about 10¹² colony forming units (cfu) of Corynebacteria spp. per dosage unit.

In another aspect, the disclosure provides kits, comprising the composition of any embodiment or combination of embodiments disclosed herein. In one embodiment, the kits may further comprise a swab or applicator. In other embodiments, dehydrated compositions of the disclosure may be present in a first container, and the kits further comprise a second container comprising a formulation for reconstituting the dehydrated Corynebacteria spp. prior to use.

### Example 1

### Summary:

Surgical site infections (SSI) are common and costly adverse events following surgical procedures, despite aseptic technique and prophylactic antibiotic use. Although gut commensals are known to limit infection in the intestine, an analogous role for skin commensals has not been described. In order to identify members of the skin microbiome with the potential to prevent infection, we characterized the wound microbiome in 49 normally healing patients undergoing skin cancer surgery. Compared to control, intact skin from the same patients, we observed striking differences in the relative abundance of particular bacterial taxa in wounds after one week of surgery. The most abundant bacteria found on intact skin, Cutibacterium acnes, was depleted in the wound microbiome. Staphylococcus aureus, a frequent cause of postoperative skin infections, made up 25% of the microbiome in normally healing wounds, suggesting active suppression of this pathogen. Finally, members of the genus Corynebacterium were significantly enriched in wounds, making up 36% of the average wound microbiome.

### Results

### Study Design and Subject Overview

Mohs micrographic surgery (MMS) wounds have unique properties that make them ideal for studying the microbiome in acute wounds. MMS wounds are generated in a controlled environment, unlike wounds resulting from nonsurgical trauma, and are therefore not exposed to a non-cutaneous reservoir of possible pathogens, such as the gut. In addition. many Mohs surgeons use second intention healing (SIH) (ref 9, 10) (i.e. permitting surgical wounds to heal without the wound edges being brought together with sutures) providing a unique opportunity to study acute open wounds.

The skin microbiomes of 70 patients undergoing MMS and managed by either complete or partial SIH were profiled 6-8 days after surgery. For each surgical site, a contralateral normal, intact skin site was sampled at the same time. Because subjects were significantly older than those studied in traditional microbiome studies (median age=71), we also sampled the normal skin microbiome from 10 of these subjects at the initial visit at both surgical and standardized skin sites (4 additional normal sites: glabella, ala, shin, and nasal mucosa) to better understand the baseline microbiome constitution in older patients. Subjects were excluded from downstream analyses if they received prophylactic antibiotics (7 subjects), had clinical evidence of surgical site infection (SSI) at the one-week visit (2 subjects), or if either matched contralateral control or surgical samples failed to pass quality control metrics (12 subjects). A total of 49 patients passed such quality control metrics; their characteristics are listed in Table 1. The microbiome from each swab sample was profiled using 16S rRNA sequencing of the VI-V3 region and a computational approach (see Methods) that enabled the classification of most skin bacteria down to the species level.

TABLE 1
Characteristics of study participants and surgical sites

| | |
|---|---|
| Patient and Surgical Site Variables | Value |

Demographics (n = 53 unique patients)

| Age, y, median (IQR) | 70 (18) |
|---|---|
| Sex, n (%) | |
| Male | 31 (58) |
| Female | 22 (42) |
| Race, n (%) | |
| Caucasion | 50 (94) |
| Unknown | 3 (6) |
| Ethnicity, n (%) | |
| Non-Hispanic | 48 (91) |
| Unknown | 5 (9) |
| Hispanic | 0 |
| Surgical Site Variables | |
| Anatomic location, n (%) | |
| Eyelid | 8 (15) |
| Postauricular/scalp | 6 (11) |
| Ear | 1 (2) |
| Nasal area | 15 (28) |
| Lip | 4 (8) |
| Forehead/temple | 9 (17) |
| Cheek/chin | 7 (13) |
| Neck | 2 (4) |
| Shin | 1 (2) |
| SIH type and area, n (%) | |
| Complete SIH | 8 (15) |
| Area, mm², median (IQR) | 300 (528) |
| Partial Closure | 45 (85) |
| Area, mm², median (IQR) | 32 (31.5) |

*IQR = interquartile range

Healing Wounds have a Distinct Microbiome

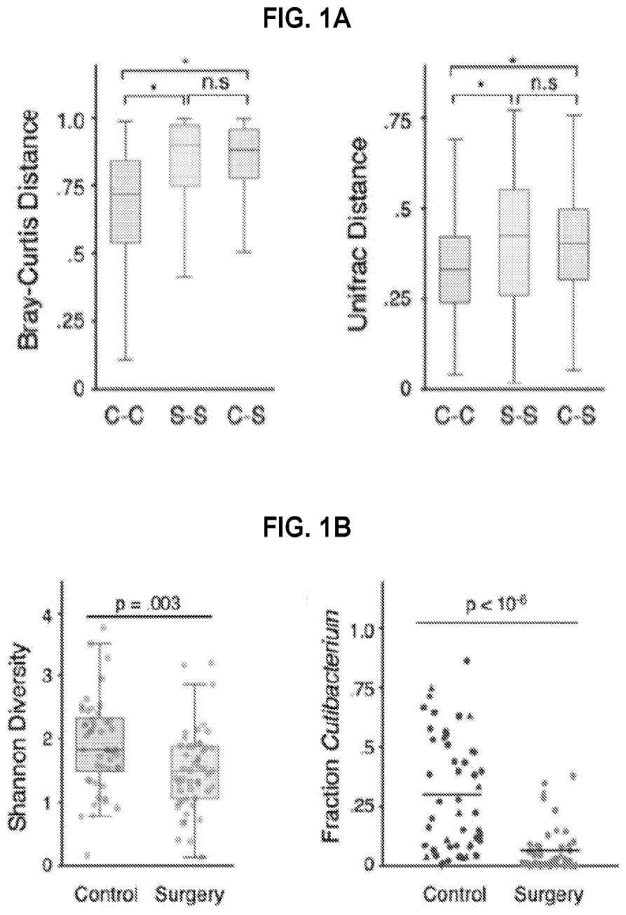

Global analysis of microbial composition shows a clear distinction between the microbiome of wounds a week after surgery and healthy contralateral skin (FIG. 1). When visualized in two-dimensions using principal-coordinate analysis, a clear clustering of wound vs. normal microbiomes is apparent (data not shown). This clustering is independent of anatomical location or the batch in which samples were processed, indicating the robust distinction between normal and healing skin microbiomes. Notably, pairs of microbiome samples from normal skin are more similar to one another than wound-normal pairs or even wound-wound pairs (FIG. 1A; P<10⁻⁶ Wilcoxon-rank-sum). Wound-wound pairs were no more similar to one another than wound-normal pairs (P=XX), suggesting that the skin microbiome of wounds can develop in diverse ways.

We find that wound skin is depleted of the Cutibacterium genus, as well as its dominant species Cutibacterium acnes, relative to control skin (FIG. 1 B; Table 2; P=<10⁻⁶, Wilcoxon-sign rank). This depletion likely reflects the surgical removal of pilosebaceous units in the wound bed, the native niche for this genus.

Conversely, wounds are enriched in the Corynebacterium genus relative to contralateral controls (P=0.004, Wilcoxon-sign-rank). Since this enrichment could have emerged as a simple artifact of relative Cutibacterium depletion, we attempted to account for the compositional nature of the data by removing all Cutibacterium from our analyses and renormalizing bacterial ratios. After this correction, Corynebacterium still remains significantly enriched in surgical wounds (Table 2; P=0.02). This suggests that the depletion of Cutibacterium bacteria in surgical wounds does not simply result in a redistribution of sequencing reads among remaining bacteria. Rather, the presence of Corynebacterium in post-surgical wounds seems to reflect that these organisms thrive in the wound-specific niche, unlike Cutibacterium.

TABLE 2
P-values for Comparison of Fractional Abundance between Wound and Matched Control Sites

| | With Cutibacterium | | | | Cutibacterium Removed | | | |
|---|---|---|---|---|---|---|---|---|
| | Mean | Mean | Fold | | Mean | Mean | Fold | |
| | Control | Wound | change on | P- | Control | Wound | change on | P- |
| | Abundance | Abundance | wounds | value* | Abundance | Abundance | wounds | value* |
| Genus | | | | | | | | |
| Cutibacterium | 0.317 | 0.063 | 0.2 | <10⁻⁶ | | | | |
| Corynebacterium | 0.181 | 0.365 | 2.0 | 0.00 | 0.253 | 0.396 | 1.6 | 0.013 |
| Staphylococcus | 0.255 | 0.375 | 1.5 | 0.088 | 0.377 | 0.392 | 1.0 | 0.940 |
| Species | | | | | | | | |
| Cutibacterium acnes | 0.307 | 0.060 | 0.20 | <10⁻⁶ | — | — | — | — |
| Staphylococcus aureus | 0.035 | 0.226 | 6.37 | 0.00 | 0.045 | 0.234 | 5.2 | 0.002 |
| Staphylococcus epidermidis | 0.146 | 0.114 | 0.78 | 0.04 | 0.223 | 0.120 | 0.5 | 0.004 |
| Staphylococcus capitis | 0.064 | 0.009 | 0.14 | <10⁻⁵ | 0.090 | 0.010 | 0.1 | <10⁻⁶ |
| Corynebacterium | 0.107 | 0.059 | 0.55 | 0.002 | 0.147 | 0.064 | 0.4 | <10⁻³ |
| kroppenstedtii | | | | | | | | |
| Corynebacterium | 0.044 | 0.154 | 3.47 | 0.002 | 0.061 | 0.169 | 2.8 | 0.024 |
| tuberculostearicum | | | | | | | | |
| Corynebacterium | 0.010 | 0.045 | 4.71 | 0.02 | 0.011 | 0.048 | 4.2 | 0.032 |
| accolens/fastidiosum | | | | | | | | |

*Wilcoxon rank-sum test, not corrected for multiple hypothesis testing

Staphylococcus aureus is Commonly Found in Normally-Healing Wounds after 1 Week

While we did not identify an enrichment of the genus Staphylococcus in surgical wounds compared to normal skin microbiomes in our analysis, stratifying staphylococcal species yielded significant variations between normal and wound skin microbiomes (Table 1). This result highlights the value of using 16S rRNA classifiers with species-level resolution, which we achieved by removing mislabeled sequences from public bacterial databases (Methods; PMID: 27166378).

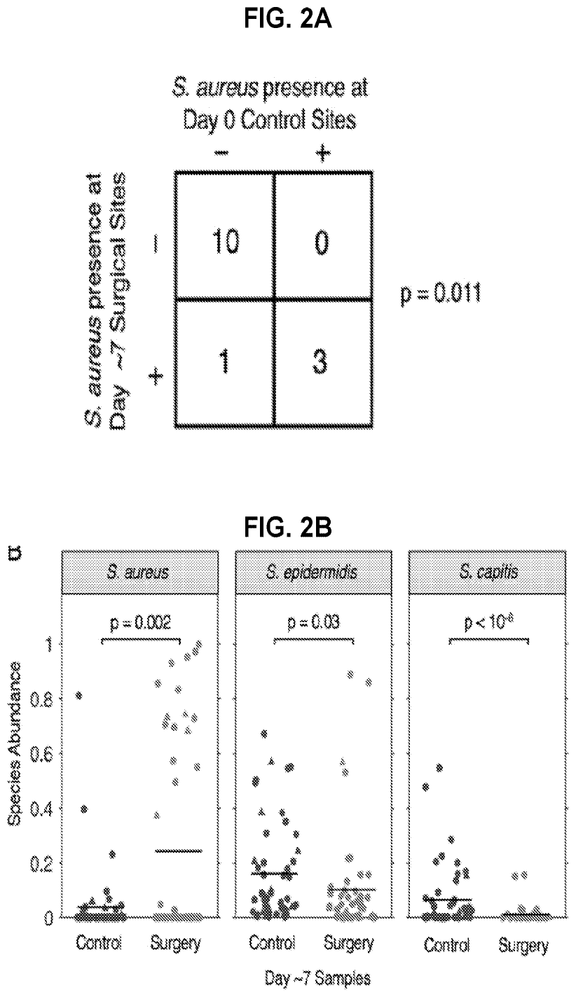

Staphylococcus epidermidis and Staphylococcus capitis are depleted on wounds relative to normal skin (P<0.03, Wilcoxon sign-rank; FIG. 2B). In contrast, S. aureus, the bacteria most commonly associated with cutaneous wound infections, is enriched in surgical sites (P<0.002; FIG. 2B). Of the 49 unique patient sample-pairs in this study, this species was found at ≥5% relative abundance in 33% of healing wounds samples. compared to only 12% of normal skin samples. As subjects with clinical signs of infections were specifically excluded from the analysis, it was surprising that S. aureus was so frequently encountered in clinically normally wound beds.

Wound colonization with S. aureus could have occurred through several mechanisms: contamination by surgical staff during the Mohs procedure, environmental contamination by patients during wound care at home, or re-implantation from the patients Staphylococcus-hosting microbiome. To distinguish between these possibilities, we leveraged the samples collected immediately after surgery in the second batch of patients—before any contaminant would have had time to expand to detectable levels. Three of the 14 patients had detectable S. aureus at one or more sampled body sites at initial sampling, and all of these three subjects went on to have detectable S. aureus on their wounds at second sampling a week later. In contrast, only one of the 11 subjects without S. aureus at the initial surgery time point had S. aureus species detected on their wounds (FIG. 2A; P=0.01, Fisher's exact test). These statistically different rates of infection support the idea that S. aureus is likely to emerge from the microbiome of each subject.

### Specific Corynebacterium Species are Enriched in Healing Wounds

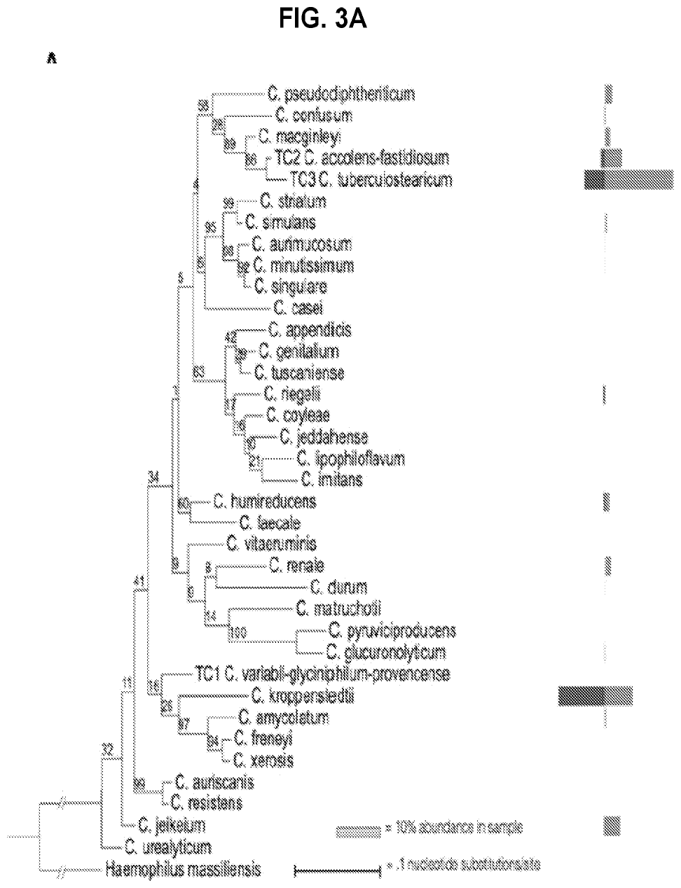

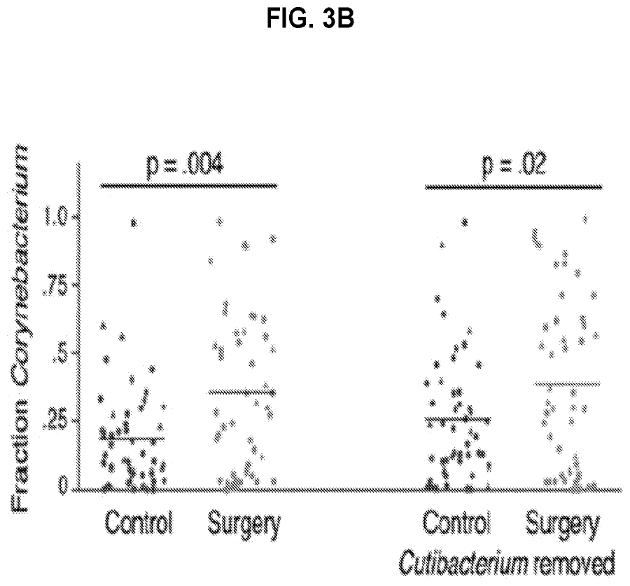

Not all Corynebacterium species found on human skin are equally enriched in wounds (FIG. 3). A diversity of type and strength of enrichments is not surprising, as Corynebacterium species comprise a diverse genus in the Actinobacteria phylum, with over 110 validated species (PMID: 29075239). Most human-associated Corynebacteria are considered commensals, commonly residing in various anatomic locations including the skin, upper respiratory tract, conjunctiva, and the urogenital tract. However, several species of Corynebacteria are known to be strictly pathogenic, most notoriously C. diphtheria (PMID: 22837327: 18275522). Many Corynebacterium species, including C. jeikeium and C. coyleae, are considered opportunistic pathogens, causing disease in patients with a history of immune compromise, malignancy, or other morbid conditions. However, the overall rarity of such infections continues to support their categorization as predominantly commensals or even contaminants (PMID: 22837327; 17992547). In our study. C. kroppenstedtii is depleted on wounds relative to healthy skin, suggesting that this species, like S. epidermidis, S. hominis, and Cutibacterium, is a poor colonizer of skin wounds.

Several Corynebacterium species are enriched on healing wounds relative to control sites, with the most significant enrichment found in C. tuberculostearicum (P<005). Other taxa enriched in wounds, though not with enough statistical enrichment to stand up to multiple hypothesis correction, include C. accolens and C. amycolatum. Interestingly, each wound tended to be dominated by just a single Corynebacterium species; the rarity of some species limited statistical power to confidently assess their ability to thrive on wounds.

### DISCUSSION

The goal of this study was to identify the bacterial inhabitants of the wound microbiome following skin surgery. We compared the microbiomes of 49 clinically non-infected surgical wounds one week after surgery to those of intact, control skin. We find that a distinct subset of organisms from the local skin microbiome invade the wound and compete to establish the new wound microbiome. The acute wound microbiome signature is marked by a depletion of Cutibacterium and an enrichment of S. aureus and Corynebacterium. The loss of Cutibacterium in the wound microbiome is predictable, as this genus primarily resides in sebaceous glands, which are removed during Mohs surgery. In contrast, S. aureus and several Corynebacterium species appear to be particularly avid colonizers of surgical wounds. These findings were enabled by a large sample size, the use of contralateral controls from the same subjects, and a species-level 16S rRNA classifier, and have implications for our understanding of colonization resistance in the skin.

It is generally understood that wound contamination by a potential pathogen can overwhelm local host defenses to cause infection, a notion that can be traced back to the origin of the germ theory and substantiated by the success of antiseptic surgical technique by Lister in the 19th century. In contrast, the potential importance of non-pathogenic bacterial colonization of wounds has received little attention. Efforts by surgeons to minimize the risk of surgical site infection have therefore focused on strategies to create as sterile a surgical environment as possible, aggressively administering topical anti-infectives and systemic antibiotics.

Our findings show the relevance of self-contamination-subjects who had S. aureus at baseline were more likely to have S. aureus colonizing their wounds a week after surgery.

Yet, the mere presence of S. aureus in a patient's wound is not sufficient for an infection to develop. Fewer than 5% of typical surgical patients develop an infection, despite our observation of S. aureus colonization in 33% of normally healing wounds (FIG. 2).

Second intention healing (STH), in which wounds remain open throughout the healing phase without surgical closure, is often deployed in dermatologic surgery,^{9,10} Infection rates from SIH are similar to infection rates after surgical wound closure and the risk of infection appears to be driven largely by anatomic site. Even the size of the open wound, and thus the time needed for completely healing, does not seem to influence infection rate.^{5,12} We hypothesize that the observed dependence of the infection rate on the anatomic site is related to the anatomic site variation of the local microbiome.

All surgical sites were prepped with 70% isopropyl alcohol prior to surgery. Sites that were partially closed were also treated with chlorhexidine prior to surgical closure. Differences were not detected between these two subsets of surgeries. The influence of these treatments on the community of free bacterial DNA diminishes on the order of hours (PMID 29753031; SanMiguel 2018); we therefore expect negligible impact remaining on the bacterial community one week later. Moreover, the relative abundance of Corynebacterium has been shown to be negatively impacted by these treatments, contrasting with and bolstering our observation of increased relative abundance of Corynebacterium after surgery. No differences were noted in the wound microbiome in lesions of different sizes, at different locations, or by closure type.

In conclusion, we observed distinct bacterial communities in acute wounds a week after surgery and obtained anatomically matched normal skin from the same patient. A surprising prevalence of S. aureus in clinically normal wounds was accompanied by outgrowth of a variety of Corynebacterium species, which modifies infection risk.

### Materials and Methods

### Study Patients

49 patients who underwent MMS with wounds managed by either partial SIH or complete SIH were recruited for this study in two batches. Swabs were obtained from the open surgical site and from the matched contralateral site during routine clinical follow-up one week (6 to 8 days) after surgery. These samples are termed batch one. To capture the microbiome on day of surgery as well as additional controls, a second study batch included additional swabs from the open wound and of the matched contralateral site on day of surgery as well as at postoperative follow-up. Additional control swabs of the nares, ala, glabella, and shin were also obtained in batch two. A swab exposed to only air was also obtained as a negative control in both phases.

### Sample Processing and Sequencing

All samples were obtained using DNA-free sterile cotton swabs that were moistened with a drop of sterile saline before sampling. Sampled surfaces were rubbed using 40 brisk strokes, placed in a sterile container, and stored at −20° C. until shipment to Microbiome Insights for processing and sequencing. A summary of the cohort characteristics is listed in Table 1.

DNA extraction, sample prep, and sequencing were performed by Microbiome Insights. DNA extraction was performed using the MoBio PowerMag™ Soil DNA Isolation Kit. PCR was performed with dual-barcoded primers (Kozich et al. 2014) targeting the 16S V1-3 (Bacteria) regions for 35 cycles. The PCR reactions were cleaned-up and normalized using the high-throughput SequalPrep™ 96-well Plate Kit and sequenced on the Illumina MiSeq™.

### 16S Amplicon Analysis

Amplicon analysis was performed using the first 180 bp after the 27F primer. Cutadapt was used to trim and remove primers from reads (Callahan et al., 2016; Martin, 2011), and QIIME2 (2020.01) and DADA2 (Bolyen et al., 834 2018: Martin 2011) were used to denoise raw reads, resulting in a table of amplicon sequence variants (ASVs) and their abundances across samples.

To classify 16S amplicon sequence variants (ASVs) at the species level, we built a classifier using a cleaned up version of the SILVA database (version 132) and the first 180 base pairs of the V1-V3 region (Quast et al., 2013). Staphylococcus species were filtered by the methods presented in (Khadka et al., 2021), and the genuses Cutibacterium, Acidipropionibacterium, Pseudopropionibacterium and families Corynebacteriaceae and Neisseriaceae were cleaned in the database using the following filters: (i) sequences with inconsistent higher taxonomic classes were removed, (ii) sequences missing a species classification were removed. (iii) species with ≥60% similarity with other taxa were relabeled as a specific “taxa cluster”, (iv) taxonomically mislabeled sequences identified using SATIVA (Kozlov et al., 2016) with greater than 90% confidence were relabeled and sequences with below 90% confidence removed. To reduce computational load, each family or genus was run independently in SATIVA; this removed about 2% of sequences from each group. The resultant quality-controlled database was used to train a naive Bayes classifier in QIIME2.

All ASVs labeled by QIIME2 as only “Bacteria” or “Bacteria: Proteobacteria” were removed from the analysis as they were found to map to human genome regions. Additionally, reads aligning to the mislabeled ASV “Bacteria; Bacteroidetes; Bacteroidia; Flavobacteriales; Flavobacteriaceae; Salinimicrobium; uncultured Pseudomonas” were removed as suspected contamination. To remove ASVs suspected to be contamination (e.g. introduced during DNA extraction). the mean abundance in air samples was compared to the average abundance in subjects samples (at any location or time point), resulting in a contamination ratio for each ASV and batch. The empirical distributions of contamination ratios were examined. and ASVs with a contamination ratio of greater than 6 for batch one and 5 for batch two were removed from the analysis. Relative abundances were then calculated from the remaining ASVs.

Samples with greater than 500 remaining ASV counts after contamination removal were included in downstream analyses. Samples from patients who were prescribed antibiotics either due to infection or as a prophylactic measure during the study were removed from the analysis. All shin or leg samples were also removed from this study due to low biomass found across all such samples.

### Statistical Methods and Phylogenetics

For all comparisons between surgical and control sites, samples were only included if both a surgical and matched control sampling site passed the sequencing depth filter mentioned above. If a patient had multiple sites sampled, only the first was included in the matched-sample analysis to remove any patient-based bias. All matched sites are obtained from the same sampling location (ex. nose, cheek, etc.), except a matched sample from patient 19, where the control was located at the glabella and surgical at the nose due to surgery placement. All statistical tests used are mentioned in the main text and figures where they are presented.

For phylogenetic reconstruction, the full 16S sequence of all Corynebacterium species found in our dataset was pulled from the SILVA database. A phylogenetic tree was constructed in MEGAx™ using the pre-aligned sequences from SILVA and a neighbor-joining model with Tamara-Nei substitutions (PMID: 31904846). This tree was bootstrapped 1000 times to investigate certainty of branch placements.

### REFERENCES

1. Dixon AJ, Dixon MP, Askew DA. Wilkinson D. Prospective study of wound infections in dermatologic surgery in the absence of prophylactic antibiotics. Dermatol Surg Off Publ Am Soc Dermatol Surg Al. 2006; 32(6):819-826: discussion 826-827. doi:10.1111/j.1524-4725.2006.32167.x
2. Rogers H D, Desciak E B, Marcus R P, Wang S, MacKay-Wiggan J, Eliezri Y D. Prospective study of wound infections in Mohs micrographie surgery using clean surgical technique in the absence of prophylactic antibiotics. J Am Acad Dermatol. 2010: 63(5):842-851. doi:10.1016/j.jaad.2010.07.029
3. Futoryan T, Grande D. Postoperative wound infection rates in dermatologic surgery. Dermatol Surg Off Publ Am Soc Dermatol Surg Al. 1995: 21(6):509-514. doi:10.1111/j. 1524-4725.1995.tb00255.x
4 Levin E C, Chow C, Makhzoumi Z, Jin C, Shiboski S C, Arron S T. Association of Postoperative Antibiotics With Surgical Site Infection in Mohs Micrographic Surgery. Dermatol Surg Off Publ Am Soc Dermatol Surg Al. 2019; 45(1):52-57. doi:10.1097/DSS.0000000000001645
5. Schimmel J, Belcher M, Vieira C, Lawrence N, Decker A. Incidence of Surgical Site Infections in Second Intention Healing After Dermatologic Surgery. Dermatol Surg Off Publ Am Soc Dermatol Surg Al. 2020; 46(12):1492-1497. doi:10.1097/DSS.0000000000002409
6. Gaines S, Luo J N, Gilbert J, Zaborina O, Alverdy J C. Optimum Operating Room Environment for the Prevention of Surgical Site Infections. Surg Infect. 2017: 18(4):503-507. doi:10.1089/sur.2017.020
7. Rhinchart M B M, Murphy M M E, Farley M F. Albertini J G. Sterile versus nonsterile gloves during Mohs micrographic surgery: infection rate is not affected. Dermatol Surg Off Publ Am Soc Dermatol Surg Al. 2006: 32(2):170-176. doi:10.1111/j.1524-4725.2006.32031.x
8. Xia Y, Cho S, Greenway H T, Zelac D E, Kelley B. Infection rates of wound repairs during Mohs micrographic surgery using sterile versus nonsterile gloves: a prospective randomized pilot study. Dermatol Surg Off Publ Am Soc Dermatol Surg Al. 2011: 37(5):651-656. doi:10.1111/j.1524-4725.2011.01949.x
9. Ravitskiy L, Brodland D G, Zitelli J A. Cost analysis: Mohs micrographic surgery. Dermatol Surg Off Publ Am Soc Dermatol Surg Al. 2012: 38(4):585-594. doi:10.1111/j.1524-4725.2012.02341.x
10. Massey P R, Gupta S, Rothstein B E, et al. Total Margin-Controlled Excision is Superior to Standard Excision for Keratinocyte Carcinoma on the Nose: A Veterans Affairs Nested Cohort Study. Ann Surg Oncol. 2021; 28(7):3656-3663. doi:10.1245/s10434-021-09604-9
11. Mott K J, Clark D P, Stelljes L S. Regional variation in wound contraction of mohs surgery defects allowed to heal by second intention. Dermatol Surg Off Publ Am Soc Dermatol Surg Al. 2003: 29(7):712-722. doi:10.1046/j. 1524-4725.2003.29180.x
12. Moreno-Arias G A, Izento-Menezes C M, Carrasco M A, Camps-Fresneda A. Second intention healing after Mohs micrographic surgery. J Eur Acad Dermatol Venereol JEADV. 2000; 14(3):159-165. doi:10.1046/j. 1468-3083.2000.00046.x
13. Alverdy J C, Hyman N. Gilbert J. Re-examining causes of surgical site infections following elective surgery in the era of asepsis. Lancet Infect Dis. 2020; 20(3): e38-e43. doi:10.1016/S1473-3099(19)30756-X
14. Garibaldi R A, Cushing D, Lerer T. Predictors of intraoperative-acquired surgical wound infections. J Hosp Infect. 1991:18 Suppl A: 289-298. doi:10.1016/0195-6701(91)90035-7
15. Grice E A, Segre J A. The skin microbiome. Nat Rev Microbiol. 2011; 9(4):244-253. doi:10.1038/nrmicro2537
16. Linehan J L, Harrison O J, Han S-J, et al. Non-classical Immunity Controls Microbiota Impact on Skin Immunity and Tissue Repair. Cell. 2018; 172(4):784-796.e18. doi:10.1016/j.cell.2017.12.033
17. Loesche M, Gardner S E, Kalan L, et al. Temporal Stability in Chronic Wound Microbiota Is Associated With Poor Healing. J Invest Dermatol. 2017; 137(1):237-244. doi:10.1016/j.jid.2016.08.009
18. Byrd A L. Belkaid Y, Segre J A. The human skin microbiome. Nat Rev Microbiol. 2018; 16(3):143-155. doi:10.1038/nrmicro.2017.157
19. Bartow-McKenney C, Hannigan G D, Horwinski J, et al. The microbiota of traumatic, open fracture wounds is associated with mechanism of injury. Wound Repair Regen Off Publ Wound Heal Soc Eur Tissue Repair Soc. 2018: 26(2):127-135. doi:10.1111/wrr.12642
20. Holder-Murray J, Yeh A, Rogers M B, et al. Time-dependent displacement of commensal skin microbes by pathogens at the site of colorectal surgery. Clin Infect Dis Off Publ Infect Dis Soc Am. Published online Oct. 24, 2020:ciaa1615. doi:10.1093/cid/ciaa1615

### Example 2

### SUMMARY

We cultured isolates of the most promising Corynebacterium species identified in our study, C. tuberculostearicum, from the skin of healthy people and performed in vitro assays that demonstrate the capacity of pre-colonization with C. tuberculostearicum to lower the abundance of S. aureus (as measured by colony forming units, CFU) and to inhibit S. aureus quorum sensing (which regulates the expression of virulence genes; as measured by RNAIII expression).

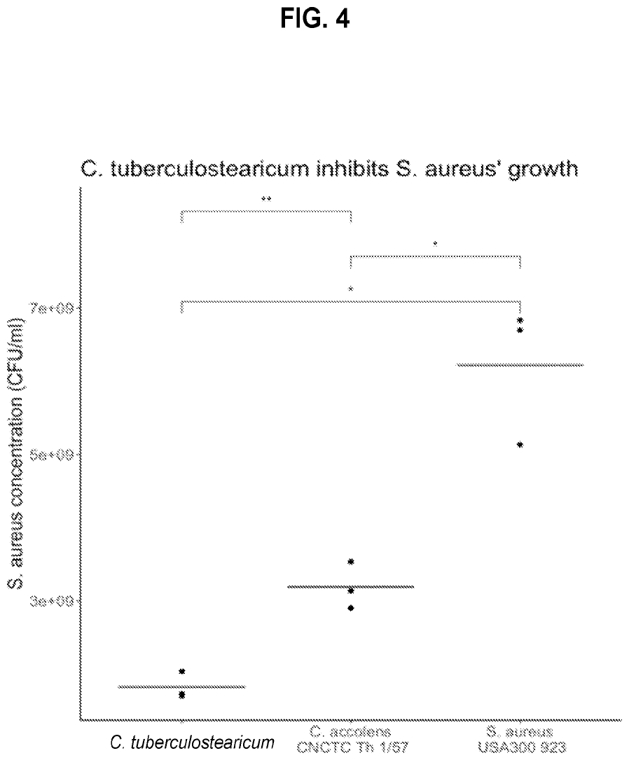

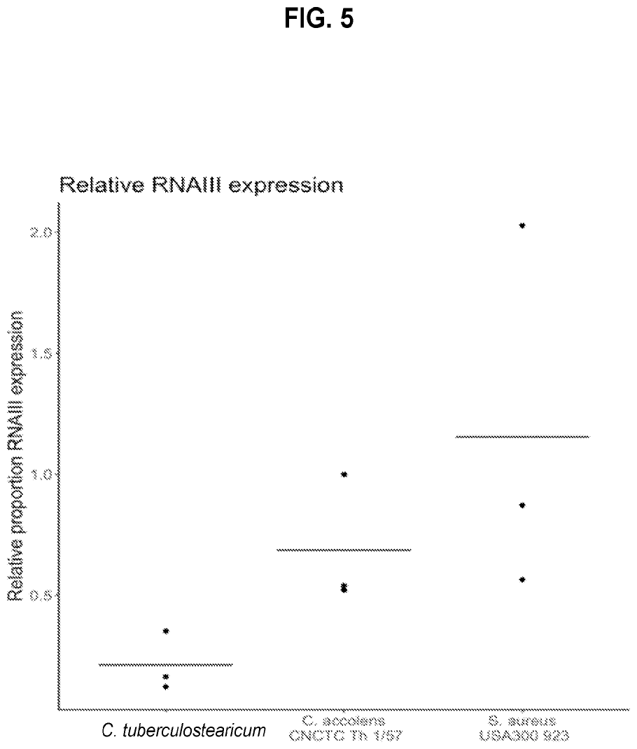

FIGS. 4 and 5 demonstrate that a focal isolate of C. tuberculostearicum significantly reduces S. aureus CFU and quorum sensing expression, respectively, and to a greater extent than C. accolens.

### Isolation:

Corynebacterium tuberculostearicum strains were isolated from face swabs gathered from healthy human subjects. Swabs taken from the forehead, chin, nose, and check were resuspended and diluted along a 10-fold dilution series. Dilutions were plated on BHI agar supplemented with 1% tween 80 and 5% defibrillated sheep blood. and incubated overnight at 37 C. Colonies were picked and identified by Sanger sequencing.

### Solid Competitions:

Saturated overnight cultures of select C. tuberculostearicum strains and a C. accolens strain were spun down and resuspended to an optical density of 0.1 in 1×PBS. Then 6 ul spots of the cultures were applied to sterile nitrocellulose membranes placed on Brain Heart Infusion plates supplemented with 5% defibrillated sheep blood and 0.25% tween 80. Control 6 ul spots of 1×PBS were applied to the membranes as well. Plates were moved to 37 C for 6h of outgrowth. Saturated cultures of S. aureus were spun down and diluted to an optical density of 0.1 in 1×PBS and then serially diluted 1:10³. Then, 2 ul of the 1:103 dilution was spotted onto the Corynebacterium and control spots, such that the S. aureus spots were fully contained within the competitor spot. Plates were returned to 37 C for 20 h. After competing, the filters were harvested into 1×PBS and vortexed to resuspend. Samples were serially diluted and plated on BHI plates for CFU enumeration. Undiluted sample was used for RNA isolation using the PURElink™ 96-well total RNA kit. Protocol was followed as written, with the exception that 4% lysostaphin was included in the lysozyme treatment and incubated with samples. cDNA was generated using the maxima H reverse transcriptase kit. qPCR was run using RNAIII and phosphate acetyltransferase (PTA) primers.

### Genomic Analysis:

C. tuberculostearicum strains were sequenced and the assembled genome for each was screened for antibiotic resistance against the Comprehensive antibiotic resistance database. The strains were classified as sensitive to all major classes of antibiotic, except Diaminopyrimidine antibiotics. One strain (DN001) is putatively resistant to Erythromycin as well.

1. A method for treating a cutaneous wound to treat or limit development of a pathogenic bacterial infection, comprising administering to a subject having a cutaneous wound an amount effective of Corynebacteria spp. to treat or limit development of pathogenic bacterial infection of the wound.

2. The method of claim 1, wherein the method treats or limits development of a pathogenic Staphylococcal infection of the wound, including but not limited to a Staphylococcal aureus infection of the wound.

3. The method of claim 1, wherein the cutaneous wound is selected from the group consisting of a penetrating wound, a puncture wound, a surgical wound or incision, a skin ulceration, a burn including but not limited to a thermal, chemical or electric burn; an insect bite or sting, a gunshot wound, or a wound caused by other high velocity projectile.

4. The method of claim 1 any one of claims 1-3, wherein the administering occurs while the wound is open, and/or after wound healing has commenced.

5. The method of claim 1, wherein the cutaneous wound is in an anatomic area with naturally low levels of Corynebacteria.

6. The method of claim 1, wherein the Corynebacteria spp. comprise one or more of C. tuberculostearicum, C. accolens, C. striatum, C. amycolatum, and/or C. pseudodipthericum.

7-10. (canceled)

11. The method of claim 1, wherein the Corynebacteria spp. is replication deficient.

12. The method of claim 1, wherein the Corynebacteria spp. is heat inactivated, UV radiation inactivated, antibiotic treatment inactivated, and/or chloroform inactivated.

13. The method of claim 1, wherein the Corynebacteria spp. are administered to the wound at between about 104 and about 1012 colony forming units (cfu).

14. The method of claim 1, wherein the Corynebacteria spp. are topically administered.

15. The method of claim 1, wherein the Corynebacteria spp. is genetically modified to inactivate one or more virulence factors and/or resistance cassettes.

16. The method of claim 1, further comprising administering antimicrobials to which Corynebacterium is resistant but S. aureus is sensitive, including but not limited to lysostaphin and mupirocin.

17. The method of claim 1, further comprising administering compounds that benefit growth of Corynebacterium, including but not limited to oleic acid and/or fructose.

18. The method of claim 1, wherein the Corynebacteria spp. is administered in a pharmaceutical composition comprising one or both of 10-50% glycerol and/or chitosan.

19. The method of claim 1, wherein the subject is a human.

20. (canceled)

21. A composition comprising Corynebacteria spp.

22. The composition of claim 21, wherein the composition is formulated for topical administration.

23-24. (canceled)

25. The composition of claim 21, wherein the Corynebacteria spp. are dehydrated and/or freeze dried.

26. The composition of claim 25, wherein the Corynebacteria spp. comprise one or more of C. tuberculostearicum, C. accolens, C. striatum, C. amycolatum, and/or C. pseudodipthericum.

27-34. (canceled)

35. A kit, comprising the composition of claim 21.

36-37. (canceled)
